(19) United States
(12) Patent Application Publication
(10) Pub. No.: US 2007/0005049 A1
(43) Pub. Date: Jan. 4, 2007

(54) Apparatus and Method of Treating Urinary Incontinence by Heating Urethra

(76) Inventor: Richard H. Comben, St. Paul, MN (US)

Correspondence Address: AMS RESEARCH CORPORATION, 10700 BREN ROAD WEST, MINNETONKA, MN, 55343, US

(21) Appl. No.: 11/427,671

(22) Filed: Jun. 29, 2006

Related U.S. Application Data
(60) Provisional application No. 60/695,671, filed on Jun. 30, 2005.

(51) Int. Cl. A61B 18/08

(52) U.S. Cl. 606/31; 128/898

(57) ABSTRACT

A method for treating urinary in continence, especially in women, is provided. The method involves reshaping the urethral lumen to improve coaptation, attenuating or dissipating stress-induced pressure pulses in the urethral lumen, of both. An apparatus and a system useful in the method are also provided.

### REFERENCE TO RELATED APPLICATIONS

This application claims the benefit of U.S. Provisional Application having Ser. No. 60/695,671, filed Jun. 30, 2005, the entire disclosure of which is incorporated herein by reference.

### FIELD

This invention relates to urinary incontinence and to methods and devices useful in treating the same. In particular, this invention relates to methods and devices for treating urinary incontinence, especially in females, by reforming the urethra with heat to; correct a loss of normal size or shape and thereby restore the ability of urethral tissue to properly coapt and seal to prevent urine flow; to reform the urethra to diffuse or attenuate aberrant pressure pulses; or both.

### BACKGROUND

Urinary continence involves coaptation of urethral tissue to produce a seal that prevents flow of urine from the bladder. Urinary incontinence can be caused if pressures or strains exerted on urethral tissue affect the shape or size of the urethra and the change in shape or size causes the urethra to lose the normal ability to coapt and produce a functional seal.

For urine to pass through the annular lumen passage of the urethra, both a pressure differential and an open lumen passage are necessary. Under most conditions there exists a pressure differential caused by static hydraulic pressure due to urine in the bladder. The pressure differential may be increased by stress-induced pressure pulses caused by coughing, sneezing, laughing, or other straining. When not urinating (or in the steady state condition), at least some portion of the urethra lumen has zero volume, i.e., the lumen is closed. The inner walls or surfaces of the urethra must be in intimate contact to prevent leakage. Also, the tissue at the closed portion of the urethra must exhibit enough squeeze force to overcome the fluid pressures and forces in the urethra due to anatomy and other causes.

The internal mucous membrane of the urethra (i.e., the collagenous tissue or lining of the inner wall) forms many longitudinal folds and the shape of the lumen varies from crescentic to stellate. These irregular surfaces make sealing more difficult and may require more squeeze force, smoother seal surface or both. A lack of squeeze force, a mismatch of tissue surface irregularities, or both, will allow leakage to occur, i.e., incontinence.

### SUMMARY

Selective heating and reformation of urethral lumen tissue can improve continence based on more than a single mechanism. Heating urethral tissue is believed to affect collagen in a way that produces both axial shrinkage of the urethra and radial swelling of collagenous urethral tissue or lining of the lumen. Only a minute amount of axial shrinkage or radial swelling is believed to be needed to improve continence. Examples of possible mechanisms of improving continence, by heating urethral tissue, include increasing the available squeeze force for sealing; smoothing the tissue sealing interfaces; and/or modifying flow field geometry.

Squeeze force can be increased by heat shrinking inner urethral lumen collagenous tissue. For example, if a small loose opening exists at the normal urethral closure (coaptation) area, leakage will occur. Heating the tissue surrounding the small opening will produce closure of the opening. Swelling the tissue to a degree beyond what is needed to have a zero opening will produce extra squeeze force.

Alternately, or in addition, selective shrinkage of the surfaces of the inner mucous membrane may make the sealing surfaces less irregular, that is smoother, thereby enhancing sealing.

Optionally, only a portion of the perimeter or circumference of coapting urethral tissue or surface, as opposed to the entire perimeter of coapting urethral tissue, may be treated with heat to improve seal performance. For example, if a portion of a perimeter of urethral tissue is damaged or misshapen, heat treatment of only a portion of the urethra, e.g., a damaged portion or an undamaged portion, may improve continence.

In other embodiments of the invention, multiple segments along a length of a urethra may be treated to achieve desired results. For example, during a coughing episode, stress-induced pressure pulses may propagate from the bladder down the liquid in the urethra, forcing the sealed junction (i.e., the coaptation area) apart. Alternatively, liquid momentum in the bladder neck could force the walls apart. If the bladder neck area acts as a diffuser, significant pressure rises may also take place at the sealing junction. Thus, according to certain methods of treatment, one or more segments along the length of a urethra may be heat treated to prevent, dissipate or attenuate pressure pulses, pressure rises, etc., from causing leakage. For example, a segment of urethral tissue at or near the bladder neck may be heat treated to produce a first treated segment. A second segment may be treated at a mid-urethra location. Reformation in this manner along the length of the urethra can create discontinuities that diffuse or attenuate coughing signatures by interfering with the propagation of the pressure pulse. They may also reduce other possible causes of leakage.

Users of the present invention will understand that treatment resulting in too high of a urethral squeeze force or too small of a lumen passage may interfere with normal or acceptable urination by potentially creating diminished urine flow during micturation. In swelling the tissue and decreasing the potential orifice size, or increasing the urethra squeeze force, care must be taken to avoid excessive tissue shrinkage, excessive squeeze force, and unduly diminished urine flow during micturation. It is important to recognize that the heated collagen may conform to the shape of the heated element and create an undesirable topographical geometry.

Described herein are devices and methods that may be used to evaluate and treat urinary incontinence, especially in females. According to the invention, a device such as probe, a catheter or a similar elongate device can be inserted into the urethra and used to selectively heat urethral tissue to modify geometry of the urethra and urethral lumen, and thereby improve continence.

In one aspect, the present invention provides a method of treating urinary incontinence that comprises the steps of:
a) providing a device comprising a therapeutic, energy-delivering probe;
b) positioning the probe proximate to a coaptation site in a urethral lumen, the urethral lumen comprising an inner wall of the collagenous tissue;
c) delivering energy to the probe; and
d) heating the collagenous tissue at the coaptation site for a time and to a temperature sufficient to axially shrink and radially swell the collagenous tissue at the coaptation site.

The energy may be delivered to only a selected portion or portions of the inner wall (i.e., the lining). When provided in this manner, the inner wall is selectively treated.

In another aspect, the present invention provides a method of reducing the effect of a stress-induced pressure pulse propagating down a lumen in a urethra of a patient comprising the steps of:
a) providing a device comprising a therapeutic, energy-delivering probe;
b) positioning the probe at a first location in the lumen proximate an inner wall of the urethra, the inner wall site comprising collagenous tissue;
c) delivering energy to the probe and heating the collagenous tissue for a time and to a temperature sufficient to form the lumen to a shape adequate to dissipate or attenuate the propagation of the pressure wave down the urethra.

In yet another aspect, the invention provides a method of treating urinary incontinence that comprises the steps of:
a) providing a device comprising an therapeutic, energy-delivering probe;
b) positioning the probe proximate to a coaptation site in a urethral lumen, the urethral lumen comprising an inner wall of collagenous tissue;
c) delivering energy to the probe; and
d) heating the collagenous tissue at the coaptation site for a time and to a temperature sufficient to axially shrink and radially swell the collagenous tissue at the coaptation site;
e) positioning a second therapeutic, energy-delivering probe in the lumen at a second location desired site between the coaptation site and a bladder opening; and
f) delivering energy to the second therapeutic probe at the second location and heating the collagenous tissue at the second location for a time and to a temperature sufficient to form the lumen at the second location to a shape adequate to dissipate or attenuate a pressure wave propagating from the bladder down the urethra.

The present invention also a provide devices and a system useful in the treatment of urinary incontinence. One embodiment of such a device is a probe for selectively heating the collagenous lining of a urethral lumen. The probe comprises a flexible elongate shaft, a first therapeutic energy-delivering element, and means for delivering energy to the heating element to selectively heat the collagenous lining.

A system according to the invention comprises a therapeutic, energy-delivering probe for heating the collagenous lining of a urethra lumen and a control means in communication with the probe to regulate the amount and duration of the energy delivered to the collagenous lining.

### BRIEF DESCRIPTION OF THE DRAWINGS

FIG. 1 schematically illustrates a device of the invention as installed in a patient's urethra;

FIGS. 2-5 schematically illustrate various embodiments of the invention;

FIG. 6 schematically illustrates an embodiment of a system of the invention;

FIG. 7 schematically illustrates another embodiment of the device of the invention; and

FIG. 8 schematically illustrates one embodiment of the method of the invention.

### DESCRIPTION

The inner mucous membrane (i.e., the lining) of the urethra is primarily made up of collagen tissue. Collagen tissue can shrink up to 30% when heated to a temperature of at least 65 degrees Celsius or more, e.g., to a temperature of or above 70 degrees Celsius. In normally healthy patients, coaptation appears to take place at the mid-urethra where the urethral tissue fibers primarily run parallel (i.e., axially) to the urethral lumen. When heated to at least 65 degrees Celsius, collagen shrinks axially and swells in a direction perpendicular to the primary fiber orientation (i.e., radially). This will tend to cause shortening of the urethra and swelling of the collagenous lining resulting in a decrease in the diameter of the urethral lumen. Applying heat selectively at a coaptation site or a portion thereof, e.g., at a mid-urethral location, may improve continence, e.g., by shrinking urethral tissue to smooth sealing surfaces of the urethral walls or to otherwise reduce or eliminate surface irregularities and/or openings in the urethral lumen, or by increasing sealing squeeze forces.

Generally, the invention comprises an apparatus and technique that involves a probe or catheter or similar device (probe and catheter are used interchangeably herein) having a shaft and one or multiple urethral tissue energy-delivering elements (e.g. heating elements) along the length of the shaft. When the probe is installed in the urethra, the heating element (or multiple elements) is (are) located in the urethra at a location for remodeling. The heating element can be actuated to emit heat energy for a time and a temperature to affect and re-shape urethral tissue, e.g., by heating collagen to cause urethral tissue to shrink or to recreate one or more discontinuities in the path of the lumen.

A device for treating urinary incontinence (e.g., in females) by heating and reforming urethra tissue may take different forms depending on the heating method, treatment site, and the desired post-treatment geometry. Exemplary probes can include a flexible elongate shaft that can be inserted into the urethra, with one or more heating elements along the length of the shaft, e.g., toward the distal end. The elongate shaft can be designed and prepared using materials and techniques that are useful in preparing elongate bodies for urethral catheters.

The energy-delivering element may be any form of heat generator that is useful to heat urethral tissue and that may be adapted to a catheter shaft. The heat generator can be any one or more of a laser, radio frequency (RF) probe, microwave probe, hot water element, steam element, resistance (i.e., electric) element, or the like. The heating element may include various components that allow selective heating of different portions or segments of the lining of the urethra, e.g., to produce desired geometries, and may exhibit any desired geometry, shape, size, and heating properties. Examples of features of a heating element may include an expandable element such as a balloon, a bladder, narrow elongate needles or extensions, and other metal extensions or assemblies such as expanding cages.

A heating element in the form of a balloon (or non-urinary bladder) can be expandable to a desired size in the urethral lumen, e.g., to expand urethral lumen during heat treatment. A heated fluid can be contained by the balloon, and can transfer sufficient heat energy through the balloon to produce a desired heat treatment of the collagenous tissue of the urethral lumen. The fluid can be any fluid, e.g., a liquid such as water, saline, or another biocompatible liquid. The fluid may be heated while in the balloon, e.g., by electric heating or another type of heat source, and the temperature may be monitored and controlled by a temperature sensor such as a thermocouple, also in the balloon. Pressure and volume of heated fluid in the balloon may also be monitored and controlled during heat treatment—e.g., fluid pressure can be maintained by removing heated fluid from a balloon to shrink the balloon as tissue shrinks during heat treatment.

Alternately, a heating element may include metal extensions that can be deployed to extend from the catheter shaft and contact urethral tissue. The metal extensions and assemblies can be expandable to a desired size in the urethral tissue, e.g., to displace urethral tissue. To produce certain geometries of treated urethral tissue, special heating element shapes and geometry modifiers fabricated out of special materials (e.g. stainless steel, titanium, etc.) may be useful.

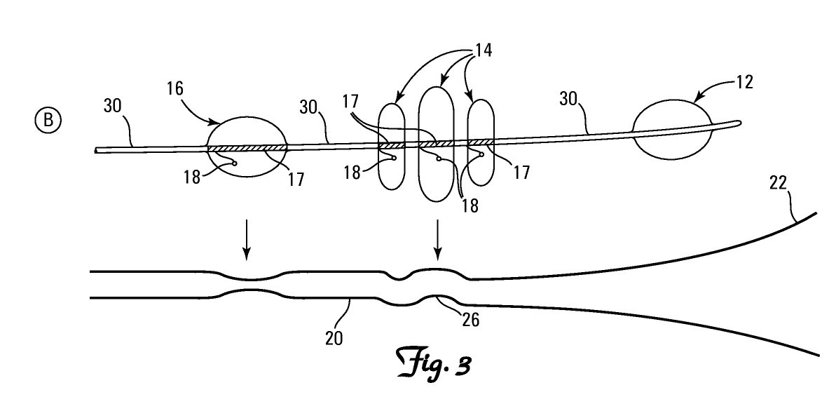

In another embodiment of a heating element, a thin and elongate, small diameter heating element may extend from the shaft, e.g., either as a single heating element or multiple elements in an array. Optionally, multiple such small diameter elongate heating elements may extend from the shaft such that they are in approximate alignment with the shaft but extend a small distance from the shaft. The heating element can be sized and shaped so that when inserted into the urethra, the small diameter heating element fits into folds or wrinkles of the urethral lumen. See, for example, FIG. 3A.

Optionally, an additional balloon (e.g., a locating balloon or end balloon) may be included at the distal end or tip of the elongate shaft of the probe. The end balloon can be used to fix the probe at a desired location during use by inflating the balloon within the bladder or bladder neck then retracting the probe to locate the balloon at the base of the bladder neck.

The attached figures illustrate various embodiments of devices of the invention. These embodiments illustrate probes that include an end balloon and multiple heating elements for treating urethral tissue (i.e., therapeutic heating elements) located along a catheter shaft proximal from the end balloon. “Proximal” refers to a location along the length of the catheter shaft that is closer to the external end of the catheter when the catheter is installed for treatment. The proximal heating elements are illustrated to be in the form of balloons, expandable metal heating elements (e.g., expandable cages), and elongate metal heating elements that may be extended from the shaft of the catheter.

Therapeutic balloons along the catheter shaft can be used to impart heat and pressure to treatment areas within the urethral lumen. If two therapeutic heating elements (e.g., balloons) are used, a first (e.g., distal) therapeutic balloon (a therapeutic balloon closest to a distal end of the device) could be used to reform urinary lumen tissue near the bladder neck to resist (i.e., dissipate or attenuate) aberrant pressures. A second, more proximal, therapeutic balloon could be used to reform urethra tissue at a coaptation or seal area.

Exemplary therapeutic energy-delivering elements can include a heating element and a temperature sensor as a part of temperature control. The energy delivered by the therapeutic element should be sufficient to produce a temperature at the mucous membrane wall that is sufficient to treat and reform urethral tissue, such as a temperature of at least 65 degrees Celsius or more, for example about 70 degrees Celsius. If the therapeutic energy-delivering probe employs or comprises a balloon, pressure within the balloon could also be monitored. The pressure could correspond to the squeeze force produced by the deformed urethra tissue. These therapeutic balloons could be non-distensible to allow more meaningful readings of the pressure in the balloon.

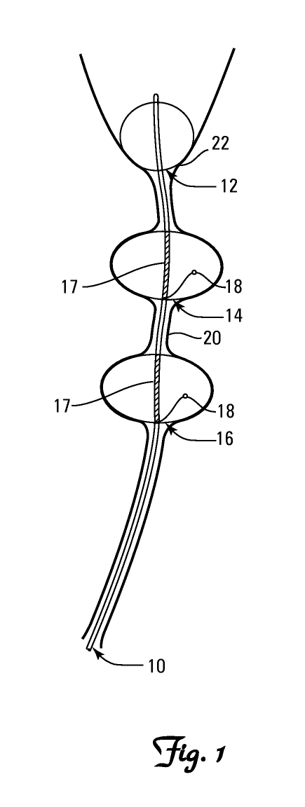

Referring to the Figures, FIG. 1 schematically illustrates a device or probe 10 of the invention as installed in a urethra 20 for heat treatment of urethral tissue. An end or “distal” balloon 12 is located in the bladder neck 22, i.e., the base of the bladder. End balloon 12, once expanded, locates the therapeutic balloon 14 at a desired location as is discussed later. It may also disrupt the flow of fluid from the bladder during the practice of the invention. Two therapeutic balloons are located at different segments along the length of the urethra. A distal therapeutic balloon 14, located closest to the bladder neck 22 and end balloon 12, is shown in an expanded state to expand urethral tissue, and is held under pressure during heat treatment.

In the method of the invention, distal balloon 14 creates an expanded section in the urethra to dissipate or attenuate the propagation of stress-induced pressure pulses from the urinary bladder down the urethra. Second therapeutic balloon 16, located more proximal to the external end of the catheter device, mid-urethra, and is shown also in an expanded state to expand urethral tissue. Second therapeutic balloon 16 creates the axial shrinkage and radial swelling in the urethra discussed above. Each of therapeutic balloons 14 and 16 includes an expandable non-urinary bladder, a heating element 17, and a temperature sensing thermocouple 18.

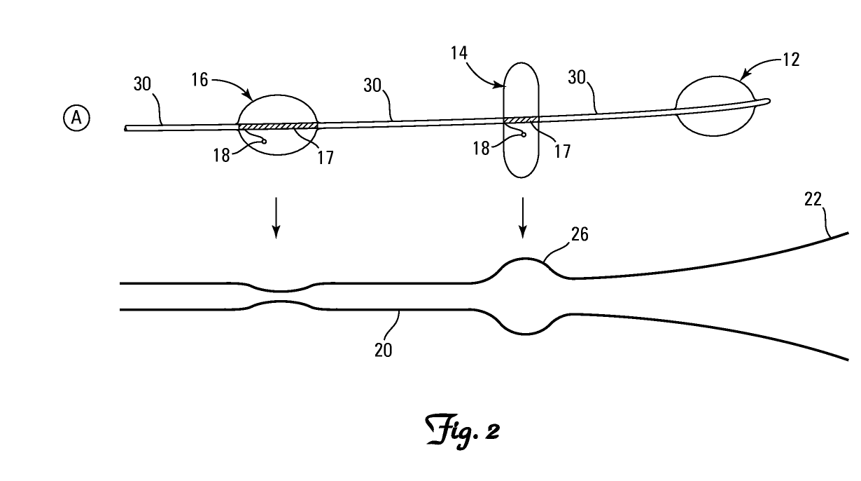

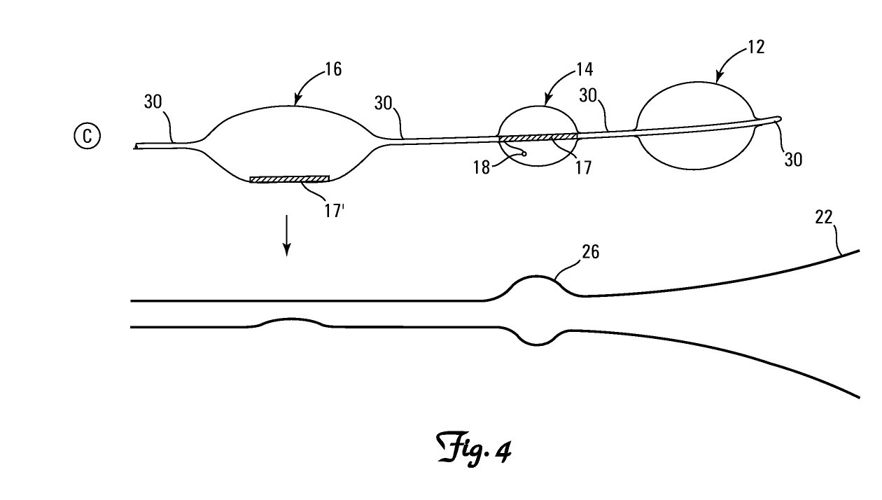

FIGS. 2 through 5 schematically illustrate alternative embodiments of devices or probes that include a catheter shaft, an end balloon, and therapeutic heating elements to heat treat different sections of length along the length of the urethra. The upper portion of each of FIGS. 2, 3 and 4, and the top and middle portions of FIG. 5 schematically illustrates a catheter shaft 30, therapeutic balloons 14 and 16, and end balloon 12.

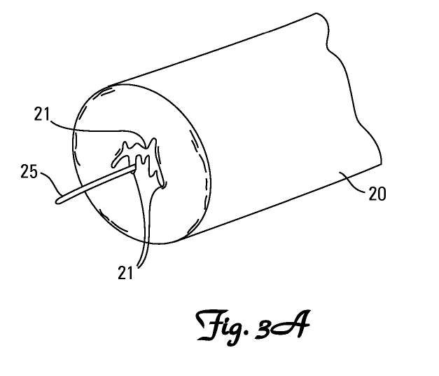

FIGS. 2-5 further illustrate that the catheter shaft has end balloon 12, first therapeutic balloon 16, second therapeutic balloon 16 (FIGS. 2, 3, 4), second therapeutic heating element 17¹, 17^{II} and 17^{III} (FIGS. 4 and 5), heating elements 17, and thermocouple 18.

The probe of FIG. 2 is similar to that of FIG. 1. The probe of FIG. 3 differs from that of FIG. 1 in that three first therapeutic balloons 14 are provided. The probe of FIG. 4 differs from that of FIG. 1 in that the second therapeutic balloon 16 has a heating element on only one side of the balloon. The probes of FIG. 5 differ from that of FIG. 1 in that each has a therapeutic heating element 17^{II} and 17^{III} that is an expanded or expandable array of heating elements. The array of elements 17^{II} comprises a longitudinal array of small diameter heaters. The array 7^{II} has a larger cross-section than that of the catheter 30. The array of elements 17^{III} comprises an expandable cage having an outer mesh 19 and struts (not shown) that support the mesh when expanded. Either of, or both of, the mesh 19 and the struts can be heaters.

FIG. 3A illustrates yet another probe useful in the present invention. FIG. 3A specifically illustrates a urethra having a lumen of generally crescentric shape. The wall of the lumen is irregular and comprises a plurality of longitudinal folds or wrinkles 21. In this embodiment of the invention, the therapeutic element 25 comprises one or more small diameter heating elements that fit into the folds 21 of the urethra 20.

The lower portion of FIGS. 3-5 schematically indicates useful locations of these elements of the catheter relative to urethral tissue during use. When the device is installed, the end balloon is located at the bladder neck 22. The first or distal therapeutic balloon 14 is at a location near the bladder neck, and a second therapeutic balloon 16 is located more proximal to the external end of the catheter device, mid-urethra. The first therapeutic balloon 14 is designed to heat treat the urethral tissue of the urethral lumen to reform a portion 26 of the length of the urethral lumen, e.g., near the bladder neck, to resist, diffuse, or attenuate pressure and flow pulses such as those that can occur during coughing or due to other stresses. As shown in FIGS. 2, 4 and 5 reformed portion 26 may be larger in cross-sectional area than the lumen. Alternatively, portion 26 may comprise a serpentine path as shown in FIG. 3. The second therapeutic balloon 16 is designed to heat treat the tissue at a mid-urethral location to reform the urethra for coaptation or sealing.

FIGS. 2 through 5 also illustrate some of the following optional features of the invention. For example, various heating elements and balloon configurations may be useful to perform selective heat treatment such as heating a portion of a total circumference or perimeter of urethral tissue, i.e., a heating element can be located only on one side of a perimeter of a catheter shaft (see, e.g., FIG. 4);

Two treatment sites may be useful, such as a treatment site at a coaptation area (normally at a mid-urethral location) and a separate treatment site distal from the coaptation area treatment site (closer to the bladder) to disrupt pressure or fluidic pulses due to coughing, etc., similar to a hydraulic attenuator.

Selective heat treatment (e.g., relative to the length of the urethra or the perimeter of the urethra) may be useful to cause an overall shrinkage of urethral tissue and still leave small areas untreated to retain tissue dynamics (see, e.g., FIGS. 3, 4, and 5).

A probe device may also include an optional vacuum feature, e.g., for use to reform tissue to improve heat transfer and move (relative to its original position) treated tissue to promote increased sealing or coaptation. The vacuum device moves tissue by collapsing the urethra so that the tissue is in improved proximity to the element.

A therapeutic balloon may be fabricated using a micro porous membrane (surface weeping), in which case a solution of lidocaine (or facsimile) may be used as an analgesic.

A heated cage-like structure (e.g., as shown in FIG. 5) may be used to produce a matrix of treated and untreated urethral tissue.

A thin and narrow (e.g., approximately 0.5 millimeter diameter) longitudinal heating element, or an array of thin and narrow heating elements, may be used to selectively treat and shrink tissue furrows in the urethra walls (e.g., as shown in FIG. 3A).

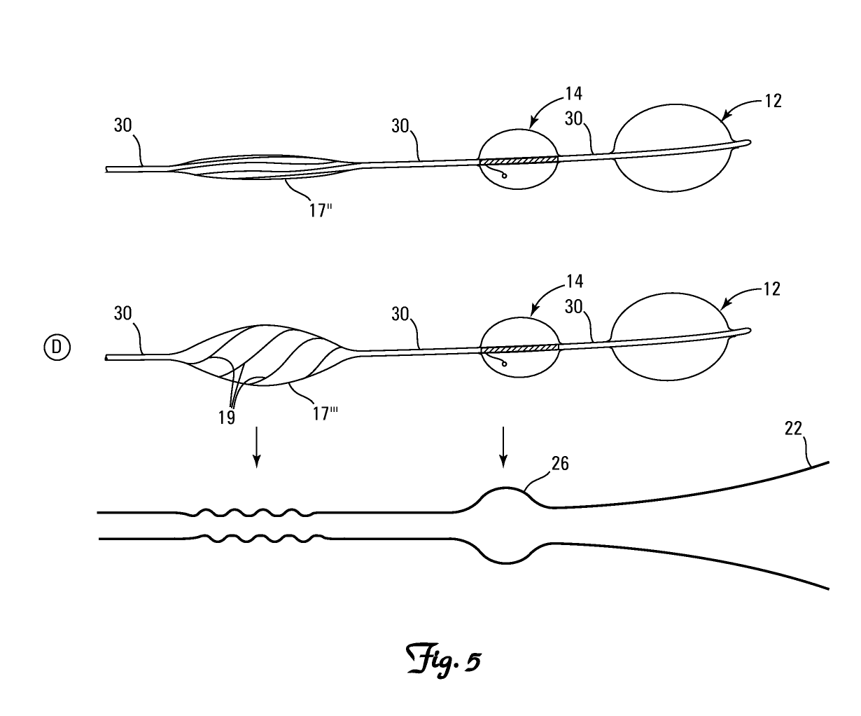

FIG. 6 illustrates a system according to the invention. The system comprises internal (distal) and external (proximal) features of an installed device. The system includes an end balloon 12 located at the distal end of a catheter device and installed at the bladder or bladder neck, and a single therapeutic balloon 16 for heat treating tissue of the urethra 20. At the proximal end is an end of an inflation lumen 40 for inflating and deflating the end balloon 12 and therapeutic balloon 16. The inflation lumen 40 terminates in an inflation coupling port 41 that receives an inflation /deflation device (not shown). A syringe 50 is shown for injecting and removing fluid into and out of the therapeutic balloon through a fluid lumen 51 to inflate and deflate the therapeutic balloon 16. Separate inflation lumens may be used to separately inflate balloons 12 and 16 if desired. A heating element (not shown) in the therapeutic balloon can heat the fluid once the fluid has been injected and the balloon has been inflated as desired. Also shown is a control module 70 that can monitor and control treatment conditions such as: temperature of the fluid in the therapeutic balloon 16; the amount (volume) of heated fluid in, and the volume of, the therapeutic balloon 16; the fluid pressure in the therapeutic balloon 16; and the residence time of the heated fluid in the therapeutic balloon 16.

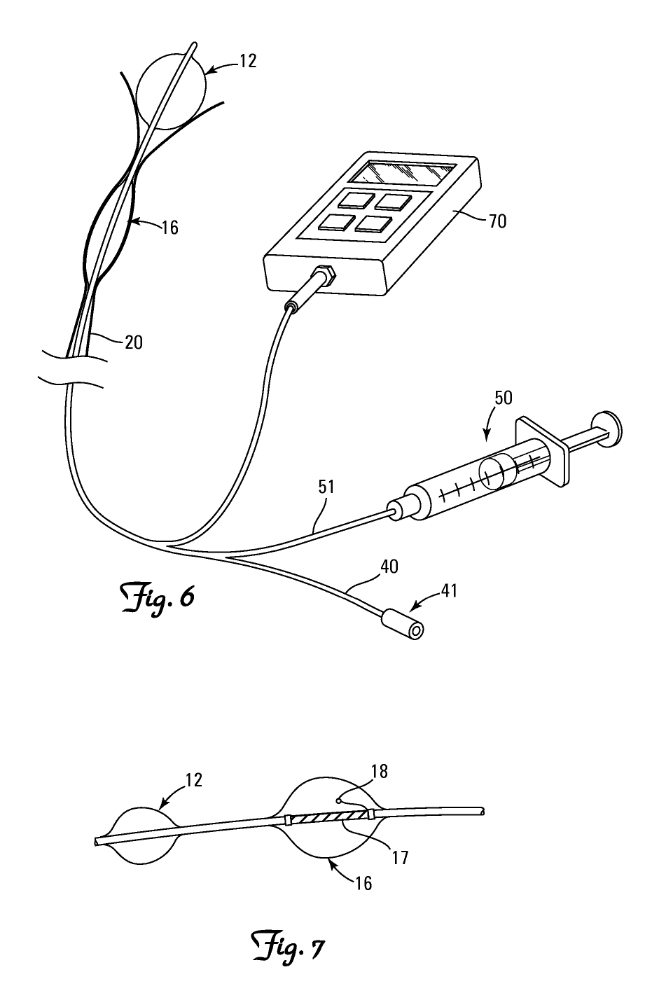

FIG. 7 illustrates details of a therapeutic balloon 16. The balloon 16 is inflated by, for example, injection of a fluid (e.g., water). A heating element 17 (e.g., an electric heating element) is wound around an internal shaft of the catheter device, and a thermocouple 18 extends from the shaft to monitor temperature of the fluid in the inflated balloon 16. Also shown in FIG. 7 is end balloon 12.

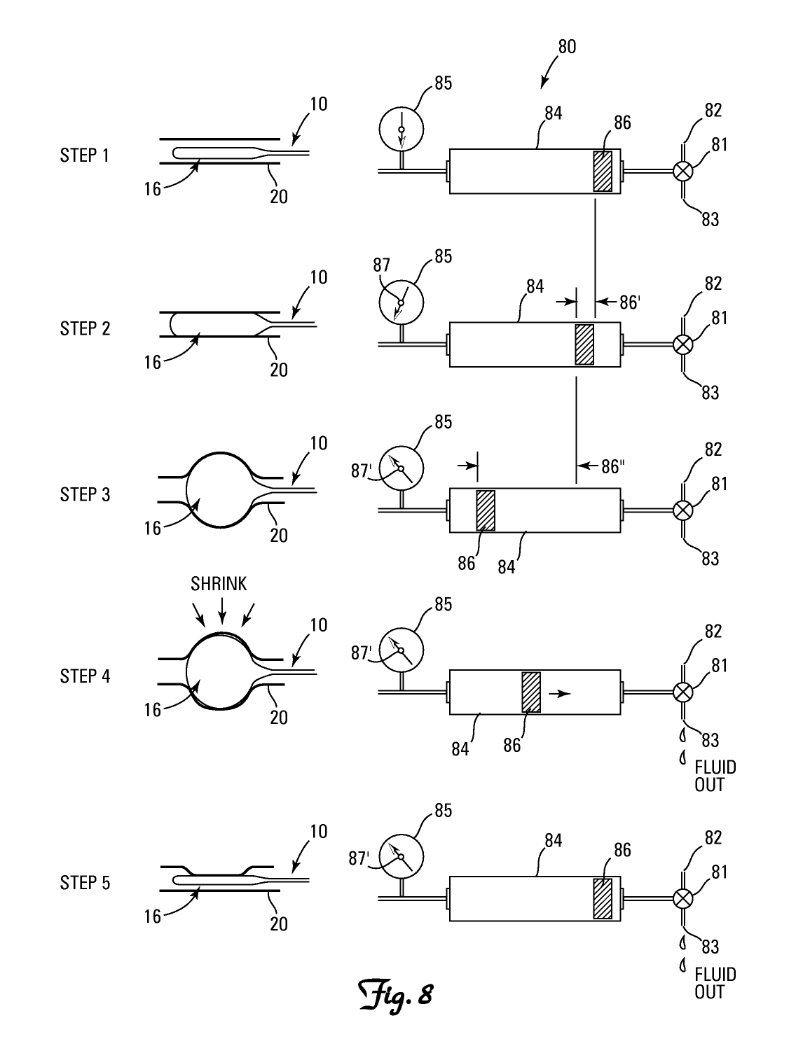

FIG. 8 illustrates selected steps from a sequence of operation of a therapeutic balloon installed at a desired location for treatment of urethral tissue, in terms of a therapeutic balloon at a mid-urethral location and an external syringe with fluid for inflating and deflating the balloon. As shown from the top to the bottom of illustrated step 1 through 5, an exemplary treatment cycle for heat treating a coaptation area of a urethra could be as follows:
At step 1, the device 10 is inserted into the urethra with a therapeutic balloon located mid-urethra. The mid-urethra is the preferred position of the balloon 16 in the method. An optional distal or end balloon (not shown) can be inflated and the catheter can be withdrawn to locate the distal balloon at the bladder neck entrance and in turn position the therapeutic balloon at the seal area (e.g., mid-urethra). An inflation device 80 is secured to the distal end of the device 10. Inflation device comprises a valve 81 that controls the flow of fluid into and out of device 10, an inlet port 82, an outlet port 83, a pressure chamber 84, and a pressure measuring means (e.g., a gauge) 85. At time zero, pressure measuring means 85 reads zero and the volume of fluid 86 (represented by a cross-hatched area) to be used to inflate balloon 60 is also zero. Inflation occurs when valve 81 is opened to allow fluid volume 86 to pass through inlet 82 into balloon 16 to push against the urethral wall. The pressure 87 in the urethra at the coaptation site (see means 85) resulting from the increased volume of fluid 86^{I} can be recorded if desired.
At step 2, the therapeutic balloon 16 is inflated until the balloon 16 begins to push at the urethra wall. This volume would be recorded.
At step 3, the balloon 16 would be further inflated by the use of an increased volume 86^{II} until it reaches an internal pressure 87^{I} of a therapeutic level (pressure exerted by a normal urethra). The increased volume of the fluid would be recorded.
Also at step 3, the fluid in the balloon would be heated and controlled via an internal heating element (not shown) and temperature sensor (e.g. thermocouple not shown).
At step 4, the heated fluid would heat the surface of the balloon and subsequently the urethral tissue (i.e., the collagenous tissue of the lining of the urethral lumen). As the heated tissue shrinks, the tissue squeezes back on the balloon (see arrows) and liquid is caused or allowed to leave the balloon through outlet 83 such that the internal pressure 87^{I,} remains at the therapeutic level and the balloon volume approaches zero.
At step 5, the end balloon (not shown) if used, is deflated and the device 10 can be removed.

A catheter device as described can also be used as a diagnostic tool. For example a collapsed balloon placed at the urethra seal area may be inflated to a therapeutic pressure level and then tested for coaptation. The volume displaced represents an approximation of the shrink volume required to maintain the therapeutic pressure or squeeze force. This therapeutic application of the device could be combined with installation of a urethral sling implant, whereby the diagnostic catheter could be used to assist in setting tension of a urethral sling.

What is claimed is:

1. A method of treating urinary incontinence comprising the steps of:
a) providing a device comprising an therapeutic, energy-delivering probe;
b) positioning the probe proximate to a coaptation site in a urethral lumen, the urethral lumen comprising an inner wall of collagenous tissues;
c) delivering energy to the probe; and
d) heating the collagenous tissue at the coaptation site for a time and to a temperature sufficient to axially shrink and radially swell the collagenous tissue at the coaptation site.

2. The method of claim 1 wherein the energy from the probe is delivered to only a portion of the collagenous tissue at the coaptation site.

3. The method of claim 1 wherein the collagenous tissue is heated to a temperature of at least 65° C. during step (d).

4. The method of claim 1 wherein the collagenous tissue axially shrinks by about at least 30% during step (d).

5. The method of claim 1 wherein the coaptation site is mid-urethra.

6. The method of claim 1 comprising the further step of applying a vacuum to the urethral lumen prior to step (c).

7. The method of claim 1 comprising the further steps of:
e) positioning a second therapeutic, energy-delivering probe in the lumen at a second location between the coaptation site and a bladder opening;
f) delivering energy to the second therapeutic probe at the second location and heating the collagenous tissue at the second location for a time and to a temperature sufficient to form the lumen at the second location to a shape adequate to cause interference, dissipate or attenuate the propagation of a pressure wave from the bladder down the urethra.

8. The method of claim 1 wherein the device further comprises a balloon surrounding the energy-delivering probe.

9. A method of reducing the effect of a stress-induced pressure pulse propagating down a lumen in a urethra of a patient comprising the steps of:
a) providing a device comprising an therapeutic, energy-delivering probe;
b) positioning the probe at a first location in the lumen proximate an inner wall of the urethra, the inner wall comprising collagenous tissue;
c) delivering energy to the probe and heating the collagenous tissue for a time and to a temperature sufficient to form the lumen to a shape adequate to dissipate or attenuate the propagation of the pressure wave down the urethra.

10. The method of claim 9 wherein the lumen is formed into a serpentine path.

11. The method of claim 9 wherein the lumen is formed into a shape having an increased cross-sectional area.

12. The method of claim 9 comprising the further step of positioning the therapeutic, energy-delivering probe at a second location in the lumen proximate the inner wall, delivering energy to the probe at the second location, and heating the collagenous tissue at the second location for a time and to a temperature sufficient to form the urethra to a shape adequate to cause further dissipation or attenuation of the propagation of the pressure wave down the urethra.

13. The method of claim 9 wherein the collagenous inner wall is heated to a temperature of at least about 65° C. during step (c).

14. A probe for selectively heating the collagenous lining of a urethral lumen comprising a flexible elongate shaft having a first therapeutic energy-delivering element at the distal end of the shaft and means for delivering energy to the heating element to selectively heat the collagenous lining.

15. The probe of claim 14 wherein the therapeutic energy-delivering element further comprises an inflatable balloon around the heating element and an inflation lumen connected to the inflatable balloon.

16. The probe of claim 14 further comprising a second therapeutic energy-delivering element.

17. The probe of claim 14 wherein the first therapeutic energy-delivering element axially shrinks and radially swells the collagenous lining and the second therapeutic energy-delivering element forms the urethral lumen to a shape adequate to dissipate or attenuate the propagation of a pressure wave from a patient's bladder down the urethra.

18. A system to treat urinary incontinence comprising:
a) a therapeutic, energy-delivering probe for heating a collagenous lining of a urethral lumen of so as to reshape the cross-section of the lumen; and
b) control means in communication with the therapeutic, energy-delivering probe to regulate the amount and duration of heat delivered to the collagenous lining.

19. The system according to claim 18 wherein the therapeutic, energy-delivering probe causes the collagenous lining to axially shrink and radially swell.

20. The system according to claim 18 wherein the therapeutic, energy-delivering probe causes the cross-sectional shape of the lumen to increase.
